(19) United States
(12) Patent Application Publication
(10) Pub. No.: US 2020/0038483 A1
(43) Pub. Date: Feb. 6, 2020

(54) MATERIAL FOR TREATMENT OF BRAIN INJURY, METHOD FOR TREATMENT OF BRAIN INJURY, MATERIAL FOR REGENERATION OF BRAIN NEURONS, AND METHOD FOR REGENERATION OF BRAIN NEURONS

(71) Applicants: Toshihiro AKAIKE, Tokyo (JP); PUBLIC UNIVERSITY CORPORATION NAGOYA CITY UNIVERSITY, Nagoya-shi (JP); NATIONAL UNIVERSITY CORPORATION TOKYO MEDICAL AND DENTAL UNIVERSITY, Tokyo (JP); SOMAR CORPORATION, Tokyo (JP)

(72) Inventors: Kazunobu SAWAMOTO, Nagoya-shi (JP); Hideo JINNOU, Nagoya-shi (JP); Masato SAWADA, Nagoya-shi (JP); Itsuki AJIOKA, Tokyo (JP); Toshihiro AKAIKE, Tokyo (JP)

(73) Assignees: PUBLIC UNIVERSITY CORPORATION NAGOYA CITY UNIVERSITY, Nagoya-shi (JP); NATIONAL UNIVERSITY CORPORATION TOKYO MEDICAL AND DENTAL UNIVERSITY, Tokyo (JP); Toshihiro AKAIKE, Tokyo (JP); SOMAR CORPORATION, Tokyo (JP)

(21) Appl. No.: 16/457,308

(22) Filed: Jun. 28, 2019

(30) Foreign Application Priority Data
Aug. 3, 2018 (JP) 2018-147211

(51) Int. Cl. A61K 38/17; A61K 47/60; A61K 47/68; A61K 47/69; A61K 9/16; A61L 27/56; A61L 27/22

(52) U.S. Cl. CPC A61K 38/1738; A61K 47/60; A61K 47/6883; A61L 27/222; A61K 9/1658; A61L 27/56; A61K 47/6939

(57) ABSTRACT

A material for treatment of brain injury, a method for treatment of brain injury, a material for regeneration of brain neurons, and a method for regeneration of brain neurons are provided. The material for treatment of brain injury contains a carrier on which at least one selected from the group consisting of N-cadherin, a fusion protein containing an entire or partial region of N-cadherin, and a fusion protein containing an entire or partial region of a protein having homology to N-cadherin is immobilized or coated.

### CROSS-REFERENCE TO RELATED APPLICATIONS

This application claims priority under 35 U.S.C. § 119 to Japanese Patent Application No. 2018-147211 filed on Aug. 3, 2018, the entire contents of which are incorporated herein by reference.

### BACKGROUND OF THE INVENTION

### 1. Field of the Invention

The present invention relates to a material for treatment of brain injury, a method for treatment of brain injury, a material for regeneration of brain neurons, and a method for regeneration of brain neurons.

### 2. Background of the Related Art

Neonatal brain injury, such as hypoxia-ischemia, is a primary cause of childhood mortality and lifelong disability. However, there is currently no therapy to repair the injured brain tissues. The “ventricular-subventricular zone (V-SVZ)” is a neural stem cell (NSC) niche in the postnatal vertebrate brain and continuously supplies neuroblasts (nerve cells) (Kaneko et al., 2017 (Non-patent Document 1)). Notably, the human neonatal V-SVZ has a remarkable neurogenic capacity (Paredes et al., 2016 (Non-patent Document 2); Sanai et al., 2011 (Non-patent Document 3)), raising the possibility that the V-SVZ could be a source for endogenous neural regeneration after neonatal brain injury.

In rodents, neuroblasts use various migratory scaffolds. In the injured adult brain, V-SVZ-derived neuroblasts migrate along blood vessels (Yamashita et al., 2006 (Non-patent Document 4)) toward the lesion. Transplanting blood-vessel-mimetic scaffolds into the injured adult brain promotes neuroblast migration to the lesion (Ajioka et al., 2015 (Non-patent Document 5); Fujioka et al., 2017 (Non-patent Document 6)). Compared to the adult brain, the neonatal brain shows migration of a larger number of neuroblasts from V-SVZ toward a lesion (Covey et al., 2010 (Non-patent Document 7)). However, the neonatal scaffolds that guide neuroblasts toward injured areas have not been fully investigated.

Radial glia are cells whose somas are located in the ventricular zone and which extend their thin fibers to the pial surface. They function as neural stem cells (NSCs) during the fetal period (Rakic, 1972 (Non-patent Document 8)). In the embryonic cerebral cortex, neuroblasts use radial glial fibers as a scaffold for migration (Kawauchi et al., 2010 (Non-patent Document 9)). In this process, radial glial fibers form adherens junction (AJ)-like structures with the neuroblasts, and guide the neuroblasts appropriately to form the cortical layers (Franco et al., 2011 (Non-patent Document 10); Rakic, 1972 (Non-patent Document 8)). Soon after birth, the radial glia transform into astrocytes or ependymal cells (Kriegstein and Alvarez-Buylla, 2009 (Non-patent Document 11)). Therefore, it remains unknown how migrating neuroblasts are guided after neonatal brain injury.

### RELATED ART DOCUMENTS, NON-PATENT DOCUMENTS

Non-patent Document 1: Kaneko et al., 2017, J. Neurochem. 141, 835-847;

Non-patent Document 2: Paredes et al., 2016, Science 354, aaf7073;

Non-patent Document 3: Sanai et al., 2011, Nature 478, 382-386;

Non-patent Document 4: Yamashita et al., 2006, J. Neurosci. 26, 6627-6636;

Non-patent Document 5: Ajioka et al., 2015, Tissue Eng. Part A 21, 193-201;

Non-patent Document 6: Fujioka et al., 2017, EBioMedicine 16, 195-203;

Non-patent Document 7: Covey et al., 2010, Dev. Neurosci. 32, 488-498;

Non-patent Document 8: Rakic, 1972, J. Comp. Neurol. 145, 61-83;

Non-patent Document 9: Kawauchi et al., 2010, Neuron 67, 588-602;

Non-patent Document 10: Franco et al., 2011, Neuron 69, 482-497; and

Non-patent Document 11: Kriegstein and Alvarez-Buylla, 2009, Annu. Rev. Neurosci. 32, 149-184.

In spite of the discoveries described above, there is no method for treatment of the brain injury at present.

In view of this, an object of the present invention is to provide a material for treatment of brain injury, a method for treatment of brain injury, a material for regeneration of brain neurons, and a method for regeneration of brain neurons.

### SUMMARY OF THE INVENTION

As described above, the radial glia normally disappear soon after birth. The present inventors discovered, however, that radial glial fibers can persist in injured neonatal mouse brains and act as a scaffold for postnatal “ventricular-subventricular zone (V-SVZ)”-derived neuroblasts that migrate to the lesion site. This injury-induced maintenance of radial glial fibers has a limited time window during postnatal development and promotes directional saltatory movement of neuroblasts via N-cadherin-mediated cell-cell contacts that promote RhoA activation. Based on these discoveries, the present inventors intensively studied to discover that transplanting an N-cadherin-containing scaffold into an injured neonatal brain similarly promotes migration and maturation of V-SVZ-derived neuroblasts, leading to functional improvements in impaired gait behaviors and hence providing solutions to the above problem, thereby completing the present invention.

That is, the present invention relates to a material for treatment of brain injury, a method for treatment of brain injury, a material for regeneration of brain neurons, and a method for regeneration of brain neurons as described below in [1] to [15].

[1] A material for treatment of brain injury, comprising a carrier on which at least one selected from the group consisting of N-cadherin, a fusion protein containing an entire or partial region of N-cadherin, and a fusion protein containing an entire or partial region of a protein having homology to N-cadherin is immobilized or coated.

[2] The material for treatment of brain injury according to [1], wherein the fusion protein containing an entire or partial region of N-cadherin, or the fusion protein containing an entire or partial region of a protein having homology to N-cadherin, has a homophilic binding capacity to N-cadherin.

[3] The material for treatment of brain injury according to [1] or [2], wherein the fusion protein containing an entire or partial region of N-cadherin, or the fusion protein containing an entire or partial region of a protein having homology to N-cadherin, is a fusion protein containing a protein selected from the following (1) to (3):

(1) N-cadherin, or a protein having an amino acid sequence having an identity of not less than 90% to N-cadherin;

(2) an extracellular domain of N-cadherin, or a protein having an amino acid sequence having an identity of not less than 90% to an extracellular domain of N-cadherin; and

(3) a protein containing one or more of EC1 domain, EC2 domain, EC3 domain, EC4 domain, and EC5 domain of N-cadherin.

[4] The material for treatment of brain injury according to any one of [1] to [3], wherein the fusion protein is a fusion protein with an Fc region of an immunoglobulin.

[5] The material for treatment of brain injury according to any one of [1] to [4], wherein the carrier is a porous body.

[6] The material for treatment of brain injury according to any one of [1] to [5], wherein the carrier is a biomaterial or biocompatible polymer carrier.

[7] The material for treatment of brain injury according to any one of [1] to [6], wherein the carrier is a biomaterial porous body.

[8] The material for treatment of brain injury according to [6] or [7], wherein the biomaterial is a protein or a polysaccharide.

[9] The material for treatment of brain injury according to any one of [6] to [8], wherein the biomaterial is gelatin or collagen.

[10] The material for treatment of brain injury according to any one of [1] to [9], wherein the carrier is a gelatin sponge.

[11] A material for regeneration of brain neurons, comprising a carrier on which at least one selected from the group consisting of N-cadherin, a fusion protein containing an entire or partial region of N-cadherin, and a fusion protein containing an entire or partial region of a protein having homology to N-cadherin is immobilized or coated.

[12] A method for treatment of brain injury, comprising transplanting the material for treatment of brain injury according to any one of [1] to [10] into a brain.

[13] The method for treatment of brain injury according to [12], wherein neuronal cells derived from pluripotent stem cells are transplanted into the brain together with the material for treatment, or after the transplantation of the material for treatment into the brain.

[14] A method for regeneration of brain neurons, comprising transplanting the material for regeneration of brain neurons according to [11] into a brain.

[15] The method for regeneration of brain neurons according to [14], wherein neuronal cells derived from pluripotent stem cells are transplanted into the brain together with the material for regeneration, or after the transplantation of the material for regeneration into the brain.

By the present invention, a material for treatment of brain injury, a method for treatment of brain injury, a material for regeneration of brain neurons, and a method for regeneration of brain neurons can be provided.

### BRIEF DESCRIPTION OF THE DRAWINGS

FIG. 1 shows diagrams illustrating that radial glia retain their fibers and provide a migratory scaffold for V-SVZ-derived neuroblasts after neonatal brain injury. (A) Experimental scheme. (B) Coronal section of the cortex in Dcx-EGFP mice stained for GFP (green) and Nestin (red) at 7 dpi. Arrowheads indicate GFP⁺ neuroblasts associated with Nestin⁺ fibers (B1 to B4). (C) Coronal section of the cortex in wild-type (WT) mice. EmGFP-expressing plasmids were electroporated into the V-SVZ, stained for GFP (green), Dcx (red), and Nestin (white). (D) Expression of N-cadherin (red) in Dcx⁺ (green) neuroblasts (asterisks) and Nestin⁺ (white) radial glial fibers (arrows). (E) Neonatal radial glial fibers after injury, targeted by adenovirus. Coronal section of the cortex in R26-tdTomato mice stained for DsRed (red) and Nestin (white). Yellow and white arrows indicate radial glial fibers located in the V-SVZ and corpus callosum (CC), respectively (E′). (F to J) Effect of expressing DN-N-cadherin (F to H) or N-cadherin-KD (I and J) in radial glial fibers on neuroblast attachment to radial glial fibers (F, G, and I) and on migration toward the lesion (F, H, and J). Coronal section of the cortex in R26-tdTomato; Dcx-EGFP mice stained for GFP (green), DsRed (red), and Nestin (white) (F). (G and I) Proportion of total neuroblasts located along radial glial fibers (whole-cell association in FIG. S2F). (K and L) TEM images of neuroblasts (N, green), control (K), and DN-N-cadherin-expressing (L) radial glial fibers (RGF, red). Red arrows and blue arrowheads indicate AJ-like electron-dense structures and irregular contacts, respectively. (M) Contact density and proportion of irregular contact regions at neuroblast-radial glial fiber adhesion points. Scale bars represent 10 μm (B), 50 μm (E), 5 μm (C, D, and F), and 500 nm (K and L). Error bars represent ±standard errors (±SEM).

FIG. 2 shows diagrams illustrating that N-cadherin promotes RhoA activation and saltatory movement in neuroblasts migrating along radial glial fibers. (A) Time-lapse images of GFP⁺ neuroblasts (green) migrating along control radial glial fibers and DN-N-cadherin-expressing tdTomato⁺ fibers (purple) in an injured cortex slice at 5 dpi. Arrows and arrowheads indicate a neuroblast's leading tip and a radial glial fibers fiber, respectively. (B to G) Migration speed (B), proportion of time spent in the radial glial fiber-attached phase (C), proportion of neuroblasts not attached to radial glial fibers (D), stride length (E), proportion of time spent in resting phase (F), and migration cycle time (G) of neuroblasts. (H and I) Time-lapse FRET ratiometric images of RhoA activity (pseudocolors) in a cultured neuroblast (H). Magnified images are shown in (I). (J) RhoA activation. (K to P) Migratory behaviors of cultured neuroblasts on N-cadherin-Fc stripes. (K) Time-lapse images of tdTomato⁺ neuroblasts (red). Migration speed (L), proportion of time spent in resting phase (M), stride length (N), and migration cycle time (O) of neuroblasts. (P) Preference for the N-cadherin-Fc stripes. Dashed lines (H and K) indicate the stripe borders. Scale bars represent 10 μm. Error bars represent ±standard errors (±SEM).

FIG. 3 shows diagrams illustrating that N-cadherin-containing scaffold promotes the migration and maturation of V-SVZ-derived neuroblasts after neonatal brain injury. (A and B) Coronal sections of the cortex in control (A) and DN-N-cadherin (B) groups stained for EmGFP (green). These are composite images of eight separate fields (two vertical and four horizontal tiles). (C) The number of EmGFP⁺NeuN⁺ cells in the injured cortex. (D) Time-lapse images of cultured neuroblasts migrating along control and N-cadherin sponge (Sp). (E) Speed of cultured neuroblasts. (F) Experimental scheme. (G) EmGFP⁺ (green) V-SVZ-derived Dcx⁺ (red) neuroblast within the N-cadherin sponge (orange). (H) Coronal sections of the cortex in wild-type mice (P2, P14, and 8 w models) treated with sponge (yellow-green), stained for Dcx (red). Arrows indicate Dcx⁺ cells along the sponge. (I) Density of Dcx⁺ cells within the sponges. (J and J′) Coronal sections of the cortex of P30 wild-type mice into which a sponge had been transplanted, stained for EmGFP (green) and NeuN (red). Arrows indicate EmGFP⁺NeuN⁺ neurons. (K) Number (left) and distribution (right) of EmGFP⁺NeuN⁺ neurons in the injured cortex. Scale bars represent 50 μm (A, B, H, and J) and 10 μm (D and G). Error bars represent ±standard errors (±SEM).

FIG. 4 shows diagrams illustrating that N-cadherin-containing scaffold improves functional recovery by promoting V-SVZ-derived neuronal regeneration after neonatal brain injury. (A to C) Catwalk analysis at P30. “Max contact area” (A), “Print area (total footprint area)” (B), and “Base of support (left anterior limb-right anterior limb distance)” (C) of the front paws. (D) Foot-fault test. Percentage of left foot faults in P2, P14, and 8 w injury models. (E) Experimental scheme. (F) Strategy for eliminating V-SVZ-derived neuroblasts. (G) Number of EmGFP⁺NeuN⁺ neuroblasts in the injured cortex at P30. (H) Foot-fault test in Ad-Cre;NSE-DTA mice into which N-cadherin sponge had been transplanted. Error bars represent ±standard errors (±SEM).

### DETAILED DESCRIPTION OF THE INVENTION

The material for treatment of brain injury of the present invention is capable of promoting migration of migrating neuroblasts, especially V-SVZ (ventricular-subventricular zone)-derived neuroblasts, toward an affected area such as a lesion, and maturation of the neuroblasts. By transplantation of the material for treatment of brain injury of the present invention into a brain, brain injury, for example, motor function that was deteriorated by brain injury can be improved.

The material for regeneration of brain neurons of the present invention is capable of promoting migration of migrating neuroblasts, especially V-SVZ (ventricular-subventricular zone)-derived neuroblasts, toward an affected area such as a lesion, and maturation of the neuroblasts to increase neurons.

The material for treatment of brain injury, the method for treatment of brain injury, the material for regeneration of brain neurons, and the method for regeneration of brain neurons of the present invention are described below in detail.

Material for Treatment of Brain Injury and Material for Regeneration of Brain Neurons

The material for treatment of brain injury and the material for regeneration of brain neurons of the present invention each comprises a carrier on which at least one selected from the group consisting of N-cadherin, a fusion protein containing an entire or partial region of N-cadherin, and a fusion protein containing an entire or partial region of a protein having homology to N-cadherin (hereinafter also simply referred to as “N-cadherin and/or the like”) is immobilized or coated.

Cadherin is an adhesion molecule involved in Ca²⁺-dependent intercellular adhesion/bonding called adhesive bonding or adherens junction, and examples of cadherin include the following three types: the E (epithelial) type, the N (neural) type, and the P (placental) type. These cadherin molecules are membrane-bound glycoprotein molecules having 700 to 750 amino acid residues whose extracellular region has five repeat structures, that is, the so-called extracellular cadherin (EC) domains, each composed of about 110 amino acid residues. For example, in a case of human N-cadherin (whose amino acid sequence is as shown in SEQ ID NO:1), EC1, EC2, EC3, EC4, and EC5 domains correspond to 160 to 267, 268 to 382, 383 to 497, 498 to 603, and 604 to 714, respectively (each numeral represents the position of a residue in the amino acid sequence of SEQ ID NO:1). In a case of mouse N-cadherin (whose amino acid sequence is as shown in SEQ ID NO:2), EC1, EC2, EC3, EC4, and EC5 domains correspond to 160 to 267, 268 to 382, 383 to 497, 498 to 603, and 604 to 717, respectively (each numeral represents the position of a residue in the amino acid sequence of SEQ ID NO:2). These EC domains have homologies between different cadherin molecule species, and the domains located in the N-terminal side (EC1 and EC2) have especially high homologies.

N-Cadherin is a protein of about 140 kD belonging to a calcium-dependent cell adhesion molecule. N-Cadherin plays an important role in cell adhesion based on interaction between the same type of cadherin and binding to the actin cytoskeleton through catenin, and is involved in development and differentiation stages. N-Cadherin is expressed in various tissues including nerves, cardiac muscle, skeletal muscle, and vascular endothelium. It has been reported that N-cadherin provides important molecular signals in a number of developmental processes including retinal development, neurogenesis, and neurite outgrowth, to function as an important regulatory factor for nervous system development (Miyatani et al., Science 1989; 245; 631-5, Hansen et al., Cell Mol. Life Sci. 2008: 65; 3809-21).

The method for preparing the N-cadherin or the fusion protein is not limited, and it is preferred to prepare and purify a recombinant protein using molecular biological techniques, and to use this protein. Any other methods may also be employed as long as a similar effect can be obtained. For example, N-cadherin may be extracted and purified from a body tissue/cells, and then be used, or its peptide may be chemically synthesized and used.

For the N-cadherin and the fusion protein, the method for preparing the recombinant protein and the method for obtaining the gene encoding the molecule may be carried out according to already established standard protocols, and an operator may refer to the above-described references. However, the methods are not limited thereto. The N-cadherin gene has already been isolated from animals such as human and mouse, and its base sequences are available in public DNA databases such as NCBI (NCBI accession number: human NM_001792, mouse NM_M31131, M22556, and the like). Thus, those skilled in the art can design a primer or a probe specific to the N-cadherin gene, and can use common molecular biological techniques to obtain and use cDNA of the N-cadherin gene. cDNA of the N-cadherin gene can also be purchased from, for example, OriGene Technologies, Inc. [https://www.origene.com/]. The gene used is preferably derived from an animal of the same species as the subject to be treated. However, the gene may also be derived from an animal of a different species.

In a preferred example of the method for preparing the N-cadherin or the recombinant protein of the fusion protein, a gene encoding the molecule is introduced into mammalian cells such as COS cells, 293 cells, or CHO cells, followed by allowing its expression. The gene is preferably linked to a nucleic acid sequence that enables transcription and expression of genes in a wide range of mammalian cells, that is, the so-called promoter sequence, such that transcription and expression of the gene are possible under control of the promoter. The gene to be transcribed and expressed is preferably further liked to a poly(A) addition signal. Preferred examples of the promoter include promoters derived from viruses such as SV (Simian Virus) 40 virus, cytomegalovirus (CMV), or Rous sarcoma virus; β-actin promoter; and EF (elongation factor) 1α promoter.

The gene to be used for the preparation of the recombinant protein does not need to contain the entire region of a gene encoding the molecule, and may be a partial gene sequence as long as the protein or the peptide molecule encoded by the partial sequence has about the same level of adhesion activity as, or a higher adhesion activity than, that of its original molecule. For example, a protein containing EC1 to EC5 domains encoding the extracellular region may be used. In general, in a cadherin molecule, the domain positioned closest to the N-terminal side (EC1) defines the binding specificity of the molecule, that is, the homophilicity (Nose et al., Cell 61: 147, 1990). Therefore, a protein molecule which contains at least EC1, and which does not contain one or several domains other than EC1, may be prepared and used.

The fusion protein containing an entire or partial region of N-cadherin, or the fusion protein containing an entire or partial region of a protein having homology to N-cadherin, preferably has a homophilic binding capacity to N-cadherin.

The fusion protein containing an entire or partial region of N-cadherin, or the fusion protein containing an entire or partial region of a protein having homology to N-cadherin, is preferably a fusion protein containing a protein selected from the following (1) to (3):

(1) N-cadherin, or a protein having an amino acid sequence having an identity of not less than 80% (preferably not less than 85%, more preferably not less than 90%, still more preferably not less than 95%) to N-cadherin;

(2) An extracellular domain of N-cadherin, or a protein having an amino acid sequence having an identity of not less than 80% (preferably not less than 85%, more preferably not less than 90%, still more preferably not less than 95%) to an extracellular domain of N-cadherin; and (3) a protein containing one or more of EC1 domain, EC2 domain, EC3 domain, EC4 domain, and EC5 domain of N-cadherin.

The fusion protein may be a fusion protein with another protein or peptide, and may be prepared, for example, as a fusion protein with an immunoglobulin Fc region, GST (Glutathione-S-Transferase) protein, MBP (Mannnose-Binding Protein) protein, avidin protein, His (oligohistidine) tag, HA (Hemagglutinin) tag, Myc tag, VSV-G (Vesicular Stromatitis Virus Glycoprotein) tag or the like to enable simple and efficient purification of the recombinant protein by use of a protein A/G column, specific antibody column or the like. Fc-fusion proteins are especially preferred for carrying out the present invention since they show excellent adsorption to synthetic polymers such as polylactic acid and polydioxane; tissue-derived biomaterials such as collagen; and biomaterials composed of ceramics such as apatite, or of a metal such as a titanium alloy, stainless steel or the like.

A large number of genes encoding the Fc region of immunoglobulin have been isolated and identified in mammals including human. A large number of their base sequences have also been reported. For example, sequence information on base sequences including those of the Fc regions of human IgG1, IgG2, IgG3, and IgG4 are available in public DNA databases such as NCBI, wherein the base sequences of human IgG1, IgG2, IgG3, and IgG4 are deposited under accession numbers AJ294730, AJ294731, AJ294732, and AJ294733, respectively. Thus, those skilled in the art can design a primer(s) or a probe(s) specific to the Fc region, and can use common molecular biological techniques to obtain and use cDNA encoding the Fc region portion. In such a case, the gene encoding the Fc region to be used is not limited regarding the animal species and the subtype, and is preferably a gene encoding, for example, the Fc region of human IgG1 or IgG2, or mouse IgG2a or IgG2b, which shows a strong binding capacity to protein A/G. Further, there is a known method in which a mutation is introduced into the Fc region to increase the binding capacity to protein A (see Nagaoka et al., Protein Eng. 16: 243, 2003 (Non-patent Document 7)), and an Fc protein genetically modified by this method may also be used.

Examples of the method for preparing the recombinant protein include a previous paper (Yue X S et al., Biomaterials 2010; 31: 5287-96).

Further, a purified recombinant protein prepared by linking cDNA of a sequence encoding the Fc region portion of human IgG and a His-tag sequence to cDNA encoding the extracellular region of human N-cadherin to provide a fusion gene, introducing the fusion gene to mouse cells, and then allowing expression of the fusion gene (Recombinant Human N-Cadherin Fc Chimera: R&D systems), and a purified recombinant protein prepared by linking cDNA of a sequence encoding the Fc region portion of mouse IgG to cDNA encoding the extracellular region of mouse N-cadherin to provide a fusion gene, introducing the fusion gene to mouse cells, and then allowing expression of the fusion gene (Recombinant mouse N-Cadherin Fc Chimera: R&D systems) are commercially available.

The carrier is preferably a biomaterial or a biocompatible polymer. Examples of the biomaterial include, but are not limited to, proteins and polysaccharides. Examples of the proteins include gelatin and collagen. Examples of the polysaccharides include chitosan and chitin. Examples of the biocompatible polymer include polyethylene glycol and polylactic acid. As the carrier, a material used for injectable gel may also be used.

The shape and the properties of the carrier are not particularly limited, and examples of the carrier include fibers, gels, and porous bodies. The carrier is especially preferably a porous body. The porous body is preferably a sponge since neurons migrate in the porous material.

The method for producing the carrier is not particularly limited. In cases of a porous body, the carrier can be obtained by, for example, freeze-drying of a gel-like biomaterial. In cases of a gelatin sponge, the carrier can be obtained by freeze-drying of gelatin.

Examples of the method for producing the gelatin sponge include a previous paper by the present inventors (Ajioka et al., Tissue Eng. Part A 21, 193-201).

The size of the porous body is not particularly limited, and is preferably a size that allows transplantation into a brain lesion.

The shape of the porous body is not particularly limited, and is preferably a shape that allows migration of neurons.

The method for immobilizing or coating N-cadherin and/or the like on the carrier is not particularly limited, and a physical method such as adsorption or a chemical method such as covalent bonding may be applied. Because of simplicity of operation, a method by adsorption is preferred. In cases where the adhesive molecule is a protein-based or peptide-based molecule, a macromolecular compound containing a sugar chain or the like, a solution of the molecule may be brought into contact with the carrier, and then the solvent may be removed after a certain period of time, to achieve simple adsorption of the molecule. More specifically, for example, in cases where the carrier is a porous body, a solution of an adhesive molecule containing distilled water, PBS or the like as a solvent may be filtered and sterilized, and then brought into contact with a porous body such as a gelatin sponge, followed by leaving the resulting product to stand for several hours to one day and night. By this, a porous body on which the adhesive molecule is immobilized or coated can be simply obtained. The resulting porous body is preferably washed with distilled water, PBS or the like several times, and then subjected to replacement with a balanced salt solution such as PBS before use.

In cases where an antigenic molecule is preliminarily artificially added to or fused with the adhesive molecule, binding of an antibody specific to the antigenic molecule may be utilized to allow efficient modification of the base material surface with the adhesive molecule, which is more preferred. In such a case, the specific antibody needs to be immobilized or coated on the carrier in advance by a physical method such as adsorption or a chemical method such as covalent bonding. For example, in cases of a recombinant protein in which an adhesive molecule is fused with an Fc region protein of IgG, the antibody with which the carrier is to be preliminarily modified may be one that specifically recognizes the Fc region of IgG. In cases of a recombinant protein in which an adhesive molecule is fused with a protein or a tag sequence peptide, the carrier may be preliminarily modified with an antibody specific to the fused molecule.

The present invention may also be carried out by using a combination of two or more selected from the group consisting of N-cadherin, a fusion protein containing an entire or partial region of N-cadherin, and a fusion protein containing an entire or partial region of a protein having homology to N-cadherin. In such cases, solutions of the proteins may be mixed together, and the resulting mixed solution may be modified according to the method described above.

The concentration of the solution of the N-cadherin and/or the like described above needs to be appropriately determined depending on the amount of adsorption and the affinity of the protein(s), and also physical properties of the protein(s). The concentration is within the range of about 0.01 to 1000 μg/mL, preferably about 0.1 to 200 μg/mL, more preferably 1 to 50 μg/mL, most preferably 3 to 20 μg/mL.

The brain injury to be treated with the material for treatment of brain injury of the present invention is not particularly limited, and examples of the brain injury include hypoxic encephalopathy, hypoxic ischemic encephalopathy, ischemic brain injury, and brain injury due to physical damage.

Method for Treatment of Brain Injury and Method for Regeneration of Brain Neurons

The method for treatment of brain injury and the method for regeneration of brain neurons of the present invention comprise transplanting the material of the present invention into a brain. The method for the transplantation is not particularly limited. The material of the present invention may be preferably transplanted such that neuronal cells, preferably V-SVZ (ventricular-subventricular zone)-derived neuroblasts, can migrate to an affected area such as a lesion, for example, such that an area where neuroblasts are present is linked to an affected area. By transplanting the material of the present invention into a brain preliminarily, or together with, for example, neuronal cells derived from pluripotent stem cells, the material of the present invention can be allowed to act as a scaffold for the transplanted neuronal cells, so that migration of the neuronal cells and regeneration of the affected area such as a lesion can be promoted. In this process, planting the neuronal cells derived from pluripotent stem cells or the like into the brain before the plantation of the material is not preferred because efficient migration of the cells to the brain lesion becomes difficult. Examples of the pluripotent stem cells include ES cells, ntES cells, and iPS cells. Examples of the neuronal cells include neural stem cells, neural progenitor cells, neurons (nerve cells), and glial cells.

Regarding the amount of the material to be transplanted, an effective amount of the material may be transplanted depending on the size of the lesion and the like.

The transplantation of the material into the brain may be carried out using known conventional surgical means. For example, the brain may be exposed by incision, and the material may be transplanted into the lesion. In cases where the material is an injectable material such as an injectable gel, the material may be locally injected into the lesion.

The material is preferably transplanted in a clean state when it is transplanted into the brain.

The individual who receives the transplantation may be a patient with brain injury. The individual is not limited to human, and may be a non-human animal such as a mammal, bird, reptile, amphibian, or fish.

### EXAMPLES

The present invention is described below in more detail by way of Examples. However, the present invention is not limited by these Examples. Unless otherwise specified, “%” as used hereinafter is on a mass basis in all cases unless otherwise specified.

Experimental Animals

All of the experiments involving live animals were performed in accordance with the guidelines and regulations of Nagoya City University and approved by the President of Nagoya City University. Animals were housed in cages lined up with chip bedding in a controlled environment (23±1° C., 12 h light/dark cycle changed at eight o'clock) with ad libitum access to water and food (MF, Oriental Yeast Co., Ltd.) in a specific-pathogen-free facility. Wild-type (WT) ICR and C57BL6/J mice were purchased from Japan SLC. The following transgenic mouse lines were used: R26-tdTomato mice (Stock No. 7914, the Jackson Laboratory), Neurog2-d4Venus mice (Kawaue et al., 2014, Dev. Growth Differ. 56, 293-304), NSE-DTA mice (Imayoshi et al., 2008, Nat. Neurosci. 11, 1153-1161; Kobayakawa et al., 2007, Nature 450, 503-508), and Dcx-EGFP mice (Gong et al., 2003, Nature 425, 917-925) (MMRRC_000244). The R26-tdTomato and NSE-DTA lines were on a C57BL6/J genetic background. The Dcx-EGFP mouse line was intercrossed with the R26-tdTomato reporter mouse line (homozygous). Genotypes were confirmed by PCR on mouse tail clippings. Cre-mediated recombination of the lox-stop-lox cassettes by adenoviral vectors (Ad-CMV-Cre and Ad-CMV-DN-N-cadherin-IRES-Cre) in the R26-tdTomato line leads to permanent tdTomato expression ubiquitously. Cre-mediated recombination of the lox-stop-lox cassettes by Ad-CMV-Cre in the NSE-DTA line leads to permanent DTA expression under the control of the NSE gene promoter. This eliminates neuronal progenies. Mice were age-matched in each experiment. Before delactation, littermates were housed with their mother or foster mouse. After delactation, the animals were divided by gender and group-housed (up to 7 mice per cage). In experiments using adult mice, 8- week-old healthy male mice were used. In other experiments using animals, both male and female healthy mice were used. Littermates were randomly assigned to experimental groups.

Culture of V-SVZ Cells

The neonatal V-SVZ was dissected from WT ICR P0-1 pups and dissociated with trypsin-EDTA (Invitrogen). Both male and female pups were used. The cells were washed twice with L-15 medium (GIBCO) containing 40 μg/mL DNase I (Roche) and then transfected with 2 μg plasmid DNA using the Amaxa Nucleofector II system (Lonza). The transfected cells were suspended in RPMI-1640 medium (Wako), incubated for 15 min at 37° C., and allowed to aggregate, and the aggregates were then cut into blocks (150 to 200 μm in diameter), mixed with 50% Matrigel (BD Biosciences) in L-15 medium, and plated on dishes. The dishes were maintained in a humidified incubator at 37° C. with 5% CO₂. The gel containing the aggregates was cultured in serum-free Neurobasal medium (GIBCO) containing 2% B-27 supernatant (Invitrogen), 2 mM L-glutamine (GIBCO), and 50 U/mL penicillin-streptomycin (GIBCO) for 48 h.

Brain Injuries

Postnatal day 2 (P2), P4, P14, and 8-week-old mice were subjected to cryogenic injury of cerebral cortex by a known method (Ajioka et al., 2015, Tissue Eng. Part A 21, 193-201). Briefly, the mice were deeply anesthetized by spontaneous inhalation of isoflurane, and the parietal skull was exposed through a scalp incision. A metal probe (1.5-mm diameter) cooled by liquid nitrogen was stereotaxically placed on the right skull (0.5-mm anterior and 1.2-mm lateral to bregma), for 30, 60, and 120 seconds in the P2, P4, P14, and 8-week-old mice, respectively. The scalp was immediately sutured, and the mice were returned to the home cage. This procedure reproducibly yielded lesions that were 500 to 600-μm deep.

Hypoxic ischemic injury was induced in P5 mice. During surgery, the mice were deeply anesthetized by spontaneous inhalation of isoflurane. The right common carotid artery was cauterized under a dissecting microscope, followed by a 1-h recovery period, and then by systemic hypoxia (oxygen/nitrogen, 8/92%) for 20 min in a plastic box at 37° C. in a humidified atmosphere. After this procedure, the mice were returned to the home cage.

Adenoviral Vectors and RNAi Constructs

To generate pENTR4-DN-N-cadherin-IRES-Cre, the IRES-Cre fragment from pLV-CMV-tdTomato-IRES-Cre (Robel et al., 2011, J. Neurosci. 31, 12471-12482) and DN-N-cadherin fragment from pCAG-MCS2-DN-N-cadherin (Nuriya and Huganir, 2006, J. Neurochem. 97, 652-661) were amplified by PCR and inserted into the BamHI and Sa/I sites of pENTR4-H1 (manufactured by RIKEN), respectively. For N-cadherin knockdown (KD) experiments using adenoviral vectors, the target sequence of the mouse N-cadherin gene was inserted into a modified Block-iT Pol II miR RNAi expression vector containing EmGFP (Invitrogen). As a control, a lacZ target sequence was used according to a known method (Ota et al., 2014, Nat. Commun. 5, 4532). To generate pENTR-tdTomato-miR-lacZ and -N-cadherin, the fragment encoding EmGFP in the pENTR-EmGFP-RfA plasmid was removed between BspMI sites, and a tdTomato fragment amplified by PCR from ptdTomato-N1 (Clontech Laboratories) was inserted. The Gateway system (Invitrogen) was used to generate the following adenoviral vectors: pAd-CMV-DN-N-cadherin-IRES-Cre, pAd-CMV-tdTomato-miR-N-cadherin, and pAd-CMV-tdTomato-miR-lacZ. These vectors were transfected into HEK293A cells to produce adenoviral particles, according to the manufacturer's instructions (Invitrogen). Adenoviral particles were concentrated by cesium chloride density-gradient centrifugation at 25,000 g for 2 h at 4° C., followed by 30,000 g for 3 h at 4° C., in an ultracentrifuge (himac CP100WX, Hitachi). As a control for Ad-CMV-DN-N-cadherin-IRES-Cre, Ad-CMV-Cre (Vector BioLabs) was used.

For the N-cadherin knockdown experiments using electroporation, the DNA cassettes (tdTomato-miR-N-cadherin and tdTomato-miR-lacZ) were cloned into a modified pCAGGS vector using the Gateway system (Invitrogen). For other knockdown experiments (FAK-KD and L1-CAM-KD), the target sequence of the mouse FAK or L1-CAM gene was inserted into a modified Block-iT Pol II miR RNAi expression vector. The DNA cassettes were cloned into a modified pCAGGS vector using the Gateway system (Invitrogen). All plasmids were prepared using a PureLink HiPure Plasmid Maxiprep Kit (Invitrogen), and the sequences were confirmed by DNA sequencing.

Injection of Adenoviral Vectors

Since radial glial cells are located at the ventricular surface and extend a long radial fiber toward the pial surface, the injection of a small volume of Ad-Cre into the cortical surface of a reporter mouse leads to retrograde infection through the fibers. Consequently, Cre-loxP-mediated recombination results in the specific and continuous labeling of radial glial cells in the neonatal brain (Merkle et al., 2007, Science 317, 381-384). Radial glial cells were labeled using P0 R26-tdTomato; Dcx-EGFP, Dcx-EGFP, or R26-tdTomato mice according to a known method (Merkle et al., 2007, Science 317, 381-384.) with some modifications. Briefly, P0 mice were anesthetized by hypothermia (5 min) or spontaneous inhalation of isoflurane, and positioned on the platform of a stereotaxic apparatus (David Kopf Instruments) by a craniophore. After the parietal skull was exposed through a scalp incision, a 20-nL volume of adenoviral suspension was injected from straight above into the surface of the cerebral cortex, using the following stereotaxic coordinates: +0.5 mm anterior +1.0 mm lateral from bregma, and +0.3 mm deep from the skull surface. The injection was made with a beveled pulled glass micropipette (Wire troll 5 μL, Drummond Scientific Company). After injection, the scalp was immediately sutured and the mice were returned to their mothers and monitored until they had resumed nursing. To assess the neuronal maturation, a 60-nL volume of adenoviral suspension was injected into P0 mice as described above using the following stereotaxic coordinates: +0.8 mm, +0.5 mm and +0.2 mm anterior +1.0 mm lateral from bregma, and +0.3 mm deep from the skull surface, to label radial glial cells in the cortex beyond the injured region. To label V-SVZ cells, a 1 μL volume of adenoviral suspension (Ad-CMV-Cre) was injected into the lateral ventricle of P0 NSE-DTA or C57BL6/J mice as described above, using the following stereotaxic coordinates: +1.8 mm anterior, +1.1 mm lateral from lambda, and +2.0 mm deep from the skull surface. The labeling efficiencies were as follows: Control (Ad-CMV-Cre) at P2, 97.7±0.5% of Nestin⁺ fibers (n=3 mice); DN-N-cadherin at P2, 98.0±0.6% (n=3 mice); p>0.05, unpaired t test; Control (Ad-CMV-Cre) at P9, 99.2±0.2% (n=4 mice); DN-N-cadherin at P9, 99.0±0.2% (n=3 mice); p>0.05, unpaired t test; Control (Ad-tdTomato-miR-lacZ) at P9, 98.7±0.4% of Nestin⁺ fibers (n=4 mice); N-cadherin-KD at P9, 97.9±0.4% (n=4 mice); p>0.05, unpaired t test.

Postnatal Electroporation

The V-SVZ cells in P0 ICR, C57BL6/J, R26-tdTomato, and NSE-DTA mice were labeled according to a known method (Ota et al., 2014, Nat. Commun. 5, 4532) with some modifications. Briefly, the mice were anesthetized by hypothermia (5 min) or spontaneous inhalation of isoflurane and fixed to the platform of a stereotaxic injection apparatus (David Kopf Instruments) by a craniophore. A solution containing EmGFP-expressing pCAGGS plasmid (7.5 μg/μL per pup) and 0.01% fast green was injected into the lateral ventricles of the right hemisphere (1.8 mm anterior, 1.25 mm lateral to lambda, and 2.0 mm deep), and introduced into V-SVZ cells by electronic pulses (70 V, 50 msec., four times) using an electroporator (CUY-21SC; Nepagene) with a forceps-type electrode (CUY650P7). V-SVZ-labeled pups were randomly subjected to cryogenic injury of cerebral cortex and sponge transplantation. When both adenovirus injection and electroporation were performed on a mouse on the same day (P0), the adenovirus was injected first, and then electroporation was performed at least 8 hours later. The labeling efficiency of V-SVZ cells by pCAGGS-EmGFP electroporation was not statistically different between experimental groups at P2 (Control, 6.3±1.2% of V-SVZ cells, n=3 mice; Injury, 6.3±1.7% of V-SVZ cells, n=3 mice; p>0.05, unpaired t test) or at P30 (Ad-Cre;control, 2.7±0.1%, n=3 mice; Ad-Cre; NSE-DTA, 2.5±0.0%, n=3 mice; p>0.05, unpaired t test). The labeling efficiency of DCX⁺ cells by pCAGGS-EmGFP electroporation (GFP⁺DCX⁺/DCX⁺ cells) in the injured cortex at P9 was 4.0±0.7% of the DCX⁺ cells (n=5 mice). For knockdown experiments (N-cadherin-KD, FAK-KD, and L1-CAM-KD), plasmid solution (7.5 μg/μL per pup) containing 0.01% fast green was injected into the lateral ventricles of the right hemisphere (1.8 mm anterior, 1.25 mm lateral to lambda, and 2.0 mm deep), and electronic pulses (70 V, 50 msec., four times) were applied by an electroporator (CUY-21SC) with a forceps-type electrode (CUY650P7) in the dorsoventral direction.

Immunoblotting

Immunoblot analysis was performed according to a known method (Ota et al., 2014, Nat. Commun. 5, 4532). To check the knockdown efficiency of the miRNAs (N-cadherin, FAK, and L1-CAM), plasmids expressing cDNA (N-cadherin, FAK, and L1-CAM) and miRNA were co-transfected into HEK293T cells using polyethylenimine. pCAG-MCS2-HA-N-cadherin (Nuriya and Huganir, 2006, J. Neurochem. 97, 652-661) was obtained as a provided product. pEGFPC1-mouse FAK (Itoh et al., 2010, Cytoskeleton (Hoboken) 67, 297-308) was obtained as a provided product. pCMV6-mouse L1-CAM was purchased from OriGene Technologies, Inc. Forty-eight hours after transfection, the cells were lysed in lysis buffer (50 mM Tris-HCl, pH 8.0, 100 mM NaCl, 1 mM EDTA, 1% NP-40, 0.01% SDS, 10 μg/mL leupeptin). To check the expression of neuregulin-1α/1β/2, cortex tissues were dissected from WT ICR P6 (4 day post injury) mice, and homogenized in lysis buffer. The lysates were briefly sonicated and cleared by centrifugation. The proteins were separated by SDS-polyacrylamide gel electrophoresis and transferred to polyvinylidene difluoride (PVDF) membranes (Millipore). The membranes were blocked in 5% skim milk in Tris Buffered Saline (TBS) containing 0.01% Tween-20, followed by incubation with primary antibodies at 4° C. overnight, and horseradish peroxidase-conjugated secondary antibodies (Jackson ImmunoResearch Inc.) at room temperature for 1 h. Signals were detected and measured with enhanced luminal-based chemiluminescent western blotting reagent (GE Healthcare) using a cooled CCD camera (LAS 3000mini, Fujifilm). The following primary antibodies were used: rat anti-HA antibody (1:1000, Roche), mouse anti-L1-CAM antibody (1:1000, Abcam), rat anti-GFP antibody (1:1000, Nacalai Tesque), rabbit anti-Neuregulin-1α/1β/2 antibody (1:1000, Santa Cruz Biotechnology), and mouse anti-actin antibody (1:10,000, Millipore). Intensities of signal expressions were calculated using ImageJ software.

Immunohistochemical Staining

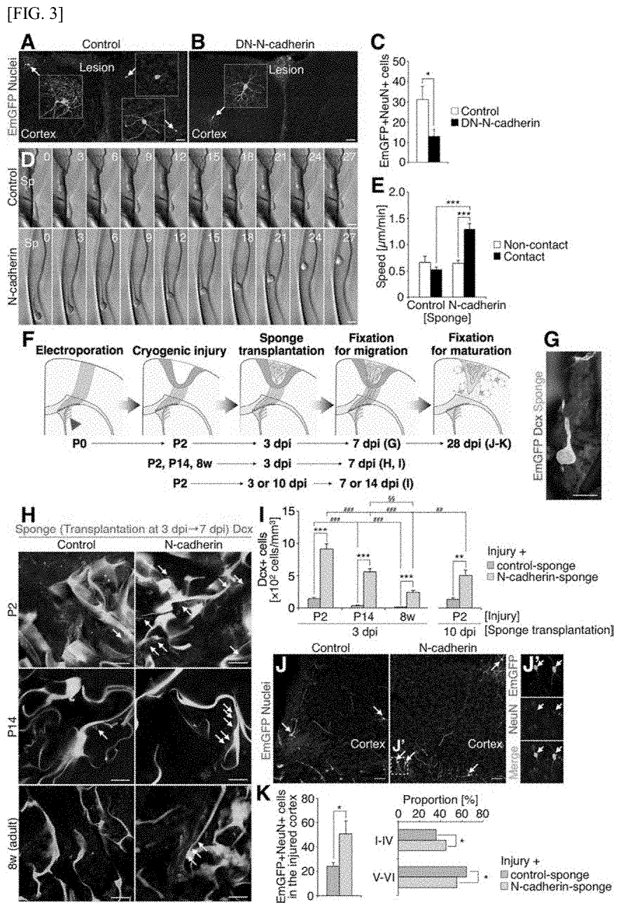

Immunohistochemical staining was performed according to a known method (Ota et al., 2014, Nat. Commun. 5, 4532). Briefly, the brain was fixed by transcardiac perfusion with 4% paraformaldehyde (PFA) in 0.1 M phosphate buffer (PB), and postfixed in the same fixative overnight at 4° C. Floating 60-μm-thick coronal sections were prepared using a vibratome sectioning system (VT1200S, Leica). The sections were incubated for 40 min at room temperature in blocking solution (10% normal donkey serum (Millipore) and 0.2% Triton X-100 in phosphate-buffered saline (PBS)), overnight at 4° C. with primary antibodies, and then for 2 h at room temperature with Alexa Fluor-conjugated secondary antibodies (1:500; Invitrogen). For the anti-Nestin antibody, AffiniPure donkey anti-chicken IgY secondary antibodies (Jackson ImmunoResearch Laboratory Inc.) were used. In the sponge transplantation experiments (FIGS. 3J, 3K, and 4G), 200-μm-thick coronal sections were treated with 100% methanol for 30 min at −30° C., acetone for 30 min at −30° C., 0.3% H₂O₂ in methanol for 2 h at room temperature, and 50% methanol for 15 min at room temperature before incubation in blocking solution (10% normal donkey serum and 0.5% Triton X-100 in PBS). Signal amplification was performed with biotinylated secondary antibodies (Jackson ImmunoResearch Laboratory Inc.) and the Vectastain Elite ABC kit (Vector Laboratories), and the signals were visualized using the TSA Fluorescence System (PerkinElmer). For Mashl staining, the sections were treated with acetone for 60 s on ice. For double staining using anti-Pax6, anti-ErbB4 or anti-Olig2 and anti-DsRed antibodies, AffiniPure Fab Fragment Donkey Anti-Rabbit IgG (H+L) (Jackson ImmunoResearch Laboratories, Inc.) was used. For double staining using anti-palvalbumin (PV), anti-calretinin (CR), or anti-GAD67 and anti-NeuN antibodies, AffiniPure Fab Fragment Donkey Anti-Mouse IgG (H+L) (Jackson ImmunoResearch Laboratories, Inc.) was used. The following primary antibodies were used: rabbit anti-Dcx (1:200, Cell Signaling Technology), guinea pig anti-Dcx (1:3000, Millipore), goat anti-Dcx antibody (1:500, Santa Cruz Biotechnology), rat anti-GFP (1:500, Nacalai), chicken anti-Nestin (1:1000, Ayes Labs), rabbit anti-DsRed (1:1000, Clontech), mouse anti-NeuN antibody (1:200, Millipore), mouse anti-CR (1:3000, Millipore), mouse anti-PV (1:2000, Sigma), mouse anti-Mashl (1:100, BD), rabbit anti-Tbr2 (1:200, Abcam), rabbit anti-Pax6 (1:100, Covance), mouse anti-N-cadherin (1:200, BD), mouse anti-glial fibrillary acidic protein (GFAP) (1:500, Sigma-Aldrich), rabbit anti-Olig2 (1:200, IBL), mouse anti-GAD67 (1:800, Millipore), rabbit anti-ErbB4 (1:300, Abcam), rabbit anti-FAK (1:100, Millipore), and mouse anti-L1-CAM (1:1000, Abcam). The guinea pig anti-D1x2 antibody (1:3000) (Kuwajima et al., 2006, J. Neurosci. 26, 5383-5392) was obtained as a provided product. For nuclear staining, Hoechst 33342 (1:3000, Thermo Fisher Scientific) was used.

Images of neuronal progenitors, radial glial fibers, mature neurons, and migrating neuroblasts associated with radial glial fibers, sponges, or polyethylene terephthalate (PET) fibers were acquired by scanning at 1 μm intervals using an LSM 700 confocal laser-scanning microscope (Carl Zeiss) with a 20× and 40× objective lens. In FIG. 3(A and B), composite images of eight separate fields (two vertical and four horizontal tiles) were acquired using the tile-scan feature of ZEN software (Carl Zeiss) with a 20× objective. To characterize the Dcx⁺, CR⁺, PV⁺, GAD67⁺, or NeuN⁺ neurons, the co-localization of signals in the cortex was confirmed by scanning at 1-μm intervals. To quantify the EmGFP⁺ cells in the V-SVZ and neuroblasts in the injured cortex, the cells were counted stereologically using a Stereo Investigator system (MBF Bioscience). After adenoviral injection and electroporation, the mice were randomly subjected to cryogenic injury of cerebral cortex and sponge transplantation. For the analyses of neuronal progenitors and migrating neuroblasts, the actual number of cells in every sixth 60-μm-thick coronal section was counted, and then the total number was estimated by multiplying the sum of the counted cells by six. To examine the radial glial fiber length and morphology, three sequential 60-μm-thick coronal sections were analyzed. In the analysis of neuroblast and radial glial fiber associations, an “association” was defined as “less than 2 μm between the neuroblast and radial glial fiber” based on previous studies (Shikanai et al., 2011, Commun. Integr. Biol. 4, 326-330). For the mature neuron analyses, all of the EmGFP⁺NeuN⁺ cells in the injured sensory and motor cortex (M2/M1/S1HL/S1FL/MPtA/LPtA/S1Tr) (Paxinos et al., 2007, J. Comp. Neurol. 145, 61-83) were analyzed. The actual number of cells in every second 60-μm-thick coronal section was counted, and then the total number was estimated by multiplying the sum of the counted cells by two. In the sponge transplantation experiments (FIGS. 3K and 4G), 200-μm-thick coronal sections were used to preserve the sponge in the injured regions. The morphology of tdTomato⁺ radial glial cells was reconstructed and quantified using Neurolucida (MBF Bioscience).

Transmission Electron Microscopy

P9 mouse brain infected with control or DN-N-cadherin-expressing adenovirus was fixed by transcardiac perfusion with 2.5% glutaraldehyde (GA) and 2% PFA in 0.1 M PB (pH 7.4). The excised brain tissue was cut into 200-μm coronal sections on a vibratome (VT1200S, Leica). The sections were treated with 2% OsO₄ in the same buffer for 2 h at 4° C. The brain tissue was then dehydrated in a graded ethanol series, placed in propyleneoxide, and embedded in Durcupan resin for 72 h at 60° C. to ensure polymerization. Semithin sections (1.5-μm-thick) were sequentially cut and stained with 1% toluidine blue, and then sections of interest were identified by light microscopy. Ultra-thin sections (60 to 70 nm) were then cut from the semi-thin sections using a high-resolution microscope (UC6, Leica) with a diamond knife, and stained with 2% uranyl acetate in distilled water for 15 min and with modified Sato's lead solution for 5 min. The sections were analyzed with a transmission electron microscope (JEM-1400plus; JEOL). The lengths of the AJ-like electron-dense adhesion structures and irregular contacts were quantified using ImageJ software (National Institutes of Health). Neuroblasts were identified by their dark cytoplasm with many free ribosomes and electron-dense nuclei, and radial glial cells were identified by their electron-lucent nuclei, and light cytoplasm with glycogen granules and abundant intermediate filaments. The numbers of analyzed cells were as follows: control, 21 cells from 2 mice; DN-N-cadherin, 17 cells from 2 mice.

Time-Lapse Imaging of Injured Brain Slices

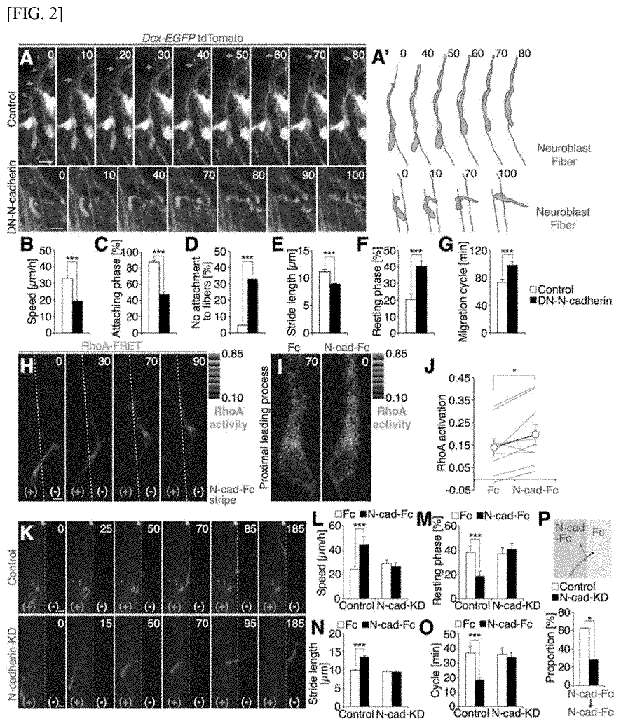

Brain slices were prepared for time-lapse imaging from neonatal 4 to 5 d-post-injury R26-tdTomato; Dcx-EGFP mice after the injection of adenoviral vectors at P0. Briefly, the brain was cut into coronal slices (200-μm thick) using a vibratome (VT1200S, Leica). The slices were placed on a stage-top imaging chamber (Warner Instruments) and kept under continuous perfusion with artificial cerebrospinal fluid (aCSF; 1 mL/min; containing 125 mM NaCl, 26 mM NaHCO₃, 3 mM KCl, 2 mM CaCl₂, 1.3 mM MgCl₂, 1.25 mM NaH₂PO₄, and 20 mM Glucose; pH 7.4; maintained at 38° C.; bubbled with 95% O₂ and 5% CO₂) during the imaging. Using a confocal laser microscope (LSM710, Carl Zeiss) equipped with a gallium arsenide phosphide detector, z stack images (4 z sections with 3 to 5-μm step sizes) were captured every 10 min for 6-16 h. The attachment time of migrating neuroblasts to radial glial fibers was evaluated as the proportion of time spent in the fiber-attached time during the migration process time. To quantify the speed, stride length, resting phase, and migration cycle of neuroblasts along radial glial fibers in captured images, neuroblasts in the cortex with a monopolar or bipolar shape were traced using ImageJ software (manual tracking plugin). The speed of the fiber extension was analyzed using ImageJ software. All of the neuroblasts that could be continuously tracked for at least 60 min were used for this analysis. For the assessment of migration cycle, all of the neuroblasts that could be continuously tracked for at least 1 cycle of saltatory movement were used. Cells in the ‘resting phase’ were defined as cells whose soma moved more slowly than 12 μm/h. In FIG. 2A, numerals indicate time (minutes) from the first frame.

Preparation of N-Cadherin-Fc-Sponge

The gelatin (GE) sponge was prepared according to a known method (Ajioka et al., 2015, Tissue Eng. Part A 21, 193-201) with modification. Fifty microliters of 3% GE beMatrix Gelatin LS-H (Nitta Gelatin) was added to each well of a 384-well plate and frozen at −20° C. The frozen GE samples were then lyophilized at 25° C., with centrifugation at 400 rpm (VC-96W; Taitec). The freeze-dried GE samples were then crosslinked with 25 mM 1-ethyl-3-(3-dimethylaminopropyl)-carbodiimide (Wako) in 90% acetone at room temperature overnight. After washing five times with double distilled water, the GE sponge was incubated in Neurobasal medium (GIBCO) for 3 h. Then, GE sponge was cut into blocks (1.2×1.2×1.2 mm³) and conjugated with 10 μg/mL N-cadherin-Fc (IgG-Fc fused with the extracellular domain of mouse N-cadherin) or Fc solutions (Yue et al., 2010, Biomaterials 31, 5287-5296) for 24 h at 4° C.

Preparation of N-Cadherin-PET Fibers

PET fibers (Inoue et al., 2009, J. Biomater. Sci. Polym. Ed. 20, 721-736), 24 μm in diameter, were obtained from Toray Industries. The PET fibers were coated with N-cadherin-Fc (IgG-Fc fused with the extracellular domain of mouse N-cadherin) or with Fc solution (Yue et al., 2010, Biomaterials 31, 5287-5296) for 1 h at 37° C., and then rinsed with PBS five times.

Transplantation of N-Cadherin-Sponges or -Fibers

N-Cadherin-Fc-sponge or control Fc-sponge, or N-cadherin-Fc-PET fibers or control Fc-PET fibers, was transplanted according to a known method (Ajioka et al., 2015, Tissue Eng. Part A 21, 193-201). Briefly, 3 or 10 days after cryogenic injury induction, the mice were anesthetized by spontaneous inhalation of isoflurane. The previous incision was cut to expose the injured parietal skull and opened by tweezers. N-Cadherin-Fc or Fc-sponge (1.2×1.2×1.2 mm³) was placed into the cavity by tweezers. In the PET-fiber transplantation, fibers at a density of about 1.2×1.2×1.2 mm³ N-cadherin-Fc- or control-Fc-fibers (1.2 mm in length) were implanted into the cavity by tweezers. After transplantation, the sponge was covered with parietal skull, and the scalp was sealed. After transplantation, the mice were placed on a warm heater to recover.

In Vitro Cell Culture

A stripe assay enabled us to analyze the migratory behaviors of single neuroblasts crossing the border between control-Fc and N-cadherin-Fc stripes. For the first stripes, 10 μg/mL N-cadherin-Fc was combined with 3 μg/mL FITC-conjugated anti-human IgG Fc antibody (Sigma) in Hank's balanced salt solution (HBSS). For the second (control) stripes, 10 μg/mL Fc was combined with 3 μg/mL anti-human-IgG Fc antibody (Sigma) in HBSS. After preincubating both stripe solutions for 30 min under moderate agitation at 4° C., 100 μL of the first stripe solution was injected into silicon matrices (50-μm wide) placed on glass-bottom 35-mm Petri dishes. After a 30-min incubation at 37° C., the dishes and matrices were rinsed with 500 μL of HBSS, and the matrices were carefully removed. The dishes were then coated with 100 μL of the second stripe solution. After a 30-min incubation at 37° C., the dishes were washed three times with HBSS. The neonatal V-SVZ was dissected from WT ICR P0-1 pups and dissociated with trypsin-EDTA (Invitrogen). The cells were washed twice with L-15 medium (GIBCO) containing 40 μg/mL DNase I (Roche) and then transfected with 2 μg of plasmid DNA (pCAGGS-tdTomato-miR-N-cadherin or -LacZ miRNA) using the Amaxa Nucleofector II system (Lonza). The transfected cells were suspended in RPMI-1640 medium (Wako) and allowed to aggregate, and the aggregates were then cut into blocks (150 to 200 μm in diameter), mixed with 50% Matrigel (BD Biosciences) in L-15 medium, and plated on the stripes.

For the neuronal culture with N-cadherin-Fc-sponge or N-cadherin-Fc-fibers, the V-SVZ cell aggregates were placed next to N-cadherin-Fc- or control-Fc-sponge, or N-cadherin-Fc- or control-Fc-fibers in 50% Matrigel. The dishes were maintained in a humidified incubator at 37° C. with 5% CO₂. The gel containing the aggregates was cultured in serum-free Neurobasal medium (GIBCO) containing 2% B-27 supernatant (Invitrogen), 2 mM L-glutamine (GIBCO), and 50 U/mL penicillin-streptomycin (GIBCO) for 48 h.

Time-lapse video recordings were obtained using an inverted light microscope (Axio-Observer, Carl Zeiss) equipped with the Colibri light-emitting diode light system, using a×20 dry objective lens. Images were obtained automatically every 3 min (FIGS. 3D and 3E) or 5 min (FIGS. 2K to 2P), for 24 h. The migration speeds were quantified using ImageJ software. All of the neuroblasts that could be continuously tracked for at least 60 min were used for this analysis. For the assessment of the migration cycle, all of the neuroblasts that could be continuously tracked for at least 1 cycle of saltatory movement were used. Cells in the ‘resting phase’ were defined as cells whose soma moved more slowly than 12 μm/h. In FIGS. 2K and 3D, numerals indicate time (minutes) from the first frame.

Immunocytochemistry

Cultured neurons on coverslips were rinsed in PBS (pH 7.4) and fixed with 4% PFA in 0.1 M PB at room temperature for 30 min. After a 40-min pre-incubation in blocking solution (10% normal donkey serum (Millipore) and 0.2% Triton X-100 in PBS), the cells were incubated with primary antibodies at 4° C. overnight. The following primary antibodies were used: rabbit anti-Dcx (1:200, Cell Signaling Technology), rabbit anti-DsRed (1:1000, Clontech), and mouse anti-N-cadherin (1:200, BD). The mouse anti-PSA-NCAM antibody (1:1000) (Seki and Arai, 1991, Neurosci. Res. 12, 503-513) was provided by Dr. Tatsunori Seki (Tokyo Medical University). For nuclear staining, Hoechst 33342 (1:3000, Thermo Fisher Scientific) was used. The multi-labeled cultured cells were analyzed with an LSM700 confocal laser-scanning microscope (Carl Zeiss), and more than three random fields were chosen under a 40× objective from each coverslip for quantification. The cell bodies of PSA-NCAM⁺ tdTomato⁺ neuroblasts were traced, and the intensity of N-cadherin expression was calculated using ZEN software (Carl Zeiss). At least three independent experiments were performed for each quantification.

FRET Imaging

FRET imaging of the RhoA activity in cultured migrating neuroblasts was performed according to a known method (Ota et al., 2014, Nat. Commun. 5, 4532). The FRET probe for RhoA (Raichu-1298X) (Yoshizaki et al., 2003, J. Cell Biol. 162, 223-232) was introduced into cultured V-SVZ-derived neuroblasts by electroporation using the Amaxa Nucleofector II system. Time-lapse imaging of the FRET-probe-expressing neuroblasts was performed using an LSM700 laser-scanning confocal microscope (Carl Zeiss) with a 40× water-immersion objective lens. The FRET ratio (intensity of FRET/CFP) was calculated, and the final images were generated using the MetaMorph software ratio image function (Molecular Devices). The baseline RhoA activity was calculated by averaging the basal activities in the leading shaft, and defining the average in each cell as 1.0. The extent of RhoA activation in the proximal leading process in a circular region of interest (ROI) (=RhoA^{prox}) was measured using the MetaMorph software Region measurements function, and normalized to the baseline activity in each frame (RhoA activation=RhoA^{prox}−1). All of the probe-expressing bipolar neuroblasts were analyzed in each experiment. Three independent experiments were performed. In FIGS. 2H and 2I, numerals indicate time (minutes) from the first frame.

Behavior Tests

Mice were subjected to quantitative neurological behavior testing at P30. The body weight was not statistically different among experimental groups. The gait behaviors on an elevated wire hexagonal grid were analyzed (Foot-fault test). In this test, the motor function involved in accurate limb placement, which is integrated with sensory feedback from the planta, is assessed (Barth et al., 1990, Behay. Brain Res. 39, 73-95). The foot-fault test was performed at 23±1° C. Briefly, mice were placed on an elevated wire hexagonal grid with 40-mm wide openings, and allowed to roam freely. A misstep was recorded as a foot fault when the mouse slipped or fell with one of its limbs dropping into an opening in the grid. The number of foot-faults for each limb was separately counted for 5 min, and then the ratio of the number of contralateral (left) fore- and hindlimb faults to the total number for the four limbs was calculated as a percentage. The test was performed twice, and the values were averaged.

Gait analysis was performed using the Noldus CatWalk XT (Noldus Information Technology), an automated gait analysis system, according to the manufacturer's instructions. Briefly, in a dark environment at 23° C.±1° C., the mice were allowed to walk across a glass walkway illuminated with a green light source. The light was completely reflected internally except at the points receiving pressure due to contact of plantae of mice with the glass surface. The contact point of each paw on the glass was illuminated, which was recorded with a high-speed video camera. The footprints recorded during each trial were analyzed using the CatWalk XT 10.5 software to generate a series of parameters. At least three successful sustained walk recordings for each mouse were used for each analysis, and the average of the runs was recorded.

Experimental Design

The number of mice, cells, and experimental replication can be found in the respective figure legend. No specific strategy for randomization was employed, and no blinding was used, except for the stereological counting of EmGFP⁺ cells in the V-SVZ and neuroblasts in the injured cortex using a Stereo Investigator system. No statistical calculation was used to estimate the sample size. Sample sizes for experiments were determined according to known studies (Ota et al., 2014, Nat. Commun. 5, 4532; Fujioka et al., 2017, EBioMedicine 16, 195-203). For analysis, animals with cryogenic injuries of cerebral cortex that were 500 to 600-μm in deep were used.

Quantification and Statistical Analysis

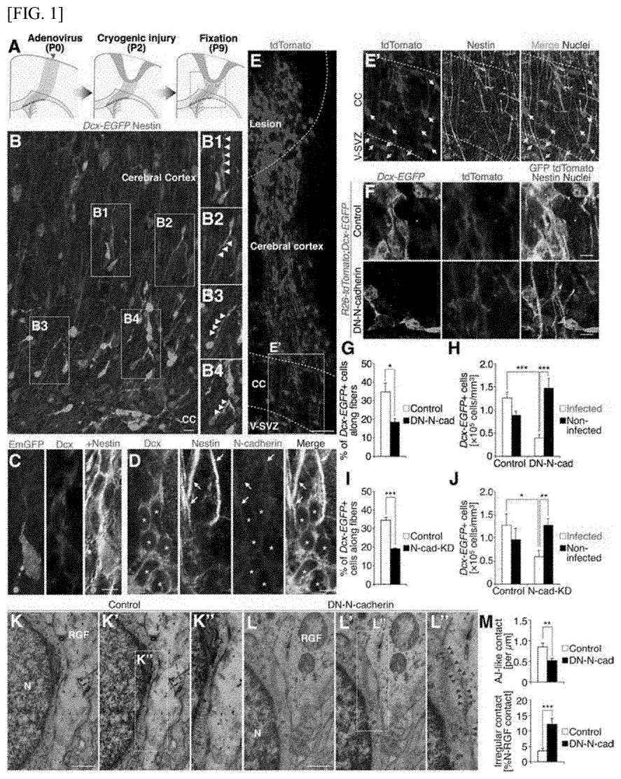

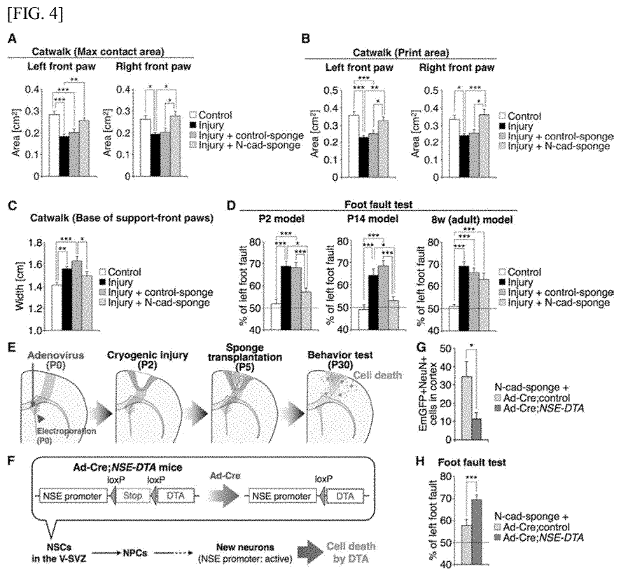

All data are shown as ±standard errors (±SEM). Two groups were compared using a two-tailed paired or unpaired t test, Wilcoxon signed-rank test, and Mann-Whitney U-test. Multiple group comparisons were performed by one-way ANOVA followed by a Tukey multiple comparison test or Dunnett test, or by a Kruskal-Wallis test followed by a Steel-Dwass multiple comparison test or Steel test. A Shapiro-Wilk test was used to assess normality. A P-value less than 0.05 was considered to be statistically significant. The statistical test used and the statistical parameters are as below: FIGS. 1G and 1I, (G) n=3 mice each; unpaired t-test, *p<0.05; (I), n=4 mice each; unpaired t-test, ***p<0.005. FIGS. 1H and 1J, (H) control, n=4 mice; DN-N-cad, n=5 mice; paired and unpaired t-test, ***p<0.005; (J) control, n=4 mice; N-cad-KD, n=4 mice; paired and unpaired t-test, *p<0.05, *p<0.01. FIG. 1M, control, n=21 cells; DN-N-cad, n=17 cells; unpaired t-test, **p<0.01, ***p<0.005. FIGS. 2B, 2E, and 2F, control, n=42 cells from 8 mice; DN-N-cad, n=60 cells from 12 mice; Mann-Whitney U-test, ***p<0.005. FIG. 2C, control, n=63 cells from 8 mice; DN-N-cad, n=107 cells from 12 mice; Mann-Whitney U-test, ***p<0.005. FIG. 2D, control, n=63 cells from 8 mice; DN-N-cad, n=107 cells from 12 mice; Fisher's exact test, ***p<0.005. FIG. 2G, control, n=39 cells from 8 mice; DN-N-cad, n=37 cells from 10 mice; Mann-Whitney U-test, ***p<0.005. FIG. 2J, n=9 cells, three independent experiments, paired t-test, *p<0.05. FIG. 2L, control, n=15 cells (five independent experiments); N-cad-KD, n=27 cells (six independent experiments), paired t-test, ***p<0.005. FIG. 2M; control, n=15 cells (five independent experiments); N-cad-KD, n=27 cells (six independent experiments), paired t-test and Wilcoxon signed-rank test, ***p<0.005. FIG. 2N, control, n=16 cells (five independent experiments); N-cad-KD, n=26 cells (six independent experiments), Wilcoxon signed-rank test, ***p<0.005. FIG. 2O, control, n=16 cells (five independent experiments); N-cad-KD, n=23 cells (five independent experiments), Wilcoxon signed-rank test, ***p<0.005. FIG. 2P, control, n=27 cells (five independent experiments); N-cad-KD, n=18 cells (four independent experiments), Chi-squared test with Yates' continuity correction. *p<0.05. FIG. 3C, control, n=6 mice; DN-N-cad, n=7 mice; unpaired t-test, *p<0.05. FIG. 3E, control-non-contact, n=14 cells; control-contact, n=19 cells; N-cad-non-contact, n=19 cells; N-cad-contact, n=28 cells; three independent experiments; unpaired t-test, ***p<0.005. FIG. 3I, P2 (3 dpi), control, n=7 mice, N-cad, n=7 mice; P14 (3 dpi), control, n=6 mice, N-cad, n=5 mice; 8 w (3 dpi), control, n=7 mice, N-cad, n=7 mice; P2 (10 dpi), control, n=4 mice, N-cad, n=5 mice; unpaired t-test, **p<0.01, ***p<0.005; control, P2 (3 dpi) vs P14 (3 dpi) or 8 w (3 dpi), one-way ANOVA followed by Tukey multiple comparison test, ###p<0.005; N-cad, (P2 [3 dpi] vs P14 [3 dpi], 8 w [3 dpi], or P2 [10 dpi], ##p<0.01, ###p<0.005), (P14 [3 dpi] vs 8 w [3 dpi], §§ p<0.01); one-way ANOVA followed by Tukey test. FIG. 3K, control, n=10 mice; N-cad, n=8 mice; left, unpaired t-test; right, Chi-squared test, *p<0.05. FIGS. 4A to 4C, n=10 mice; one-way ANOVA followed by Tukey test, except for (A) right (Kruskal-Wallis test followed by Steel-Dwass test), *p<0.05, **p<0.01, ***p<0.005. FIG. 4D, P2 model, control, n=11 mice; injury, n=10 mice; injury+control-sp, n=13 mice; injury+N-cad-sp, n=14 mice, Kruskal-Wallis test followed by Steel-Dwass test; P14 and 8 w models, n=7 mice each, one-way ANOVA followed by Tukey test. *p<0.05, ***p<0.005. FIG. 4G, control, n=5; NSE-DTA, n=4; unpaired t-test, *p<0.05. FIG. 4H, control, n=11 mice; NSE-DTA, n=7 mice; unpaired t-test, ***p<0.005. All statistical data, including the statistical tests used, standard errors (±SEM), and P values are indicated in the text, figure legends, and figures. Error bar values indicate ±standard errors (±SEM). Littermates were randomly assigned to experimental groups.

Results

Neonatal Radial Glial Cells Maintain Their Fibers After Brain Injury

Cryogenic injury of cerebral cortex was performed on postnatal day 2 (P2), and the dynamics of radial glial fiber disappearance was analyzed. In the contralateral (uninjured) side, the density of Nestin⁺ radial glial fibers gradually decreased, which was consistent with previous observations (Kriegstein and Alvarez-Buylla, 2009, Annu. Rev. Neurosci. 32, 149-184). On the other hand, the density of radial glial fibers in the cortex in the ipsilateral side (injured side) was significantly higher than that in the contralateral side at all of these time points, although it was highest at 7 day post injury (dpi) and decreased thereafter. In addition, the radial glial fibers were longer in the injured brain than in the uninjured brain. These results suggested that neonatal brain injury promotes the maintenance of radial glial fibers.

To examine the effects of an injury caused in later stages on radial glial fibers, cryogenic injury of cerebral cortex was performed in P4, P14, and 8-week-old (8 w, adult) mice, and the radial glial fibers were analyzed 7 days later. The Nestin⁺ fibers were retained in the P4 model, although the fiber density in the P4 model was significantly lower than in the P2 model. No clear Nestin⁺ radial glial fibers were observed in the P14 or 8 w injury models. These results suggested that radial glial fibers have the potential to be retained after injury only during the neonatal stages. Time-lapse imaging of cultured brain slices revealed that the diminished radial glial fibers could regrow in response to injury. Consistently, these fibers in the injured brain were significantly longer than those in the uninjured brain in the P14 and 8 w models. Radial glial fibers were also observed in the neonatal mouse brain after hypoxic and ischemic injury. Taken together, these results suggested that the neonatal brain has the potential to maintain radial glial fibers after injury.

Neonatal Radial Glial Cells Provide Migratory Scaffold for V-SVZ-Derived Neuroblasts After Brain Injury

A cryogenic injury was prepared at P2, and neurogenesis after the injury was studied at P9 (FIG. 1A). A large number of doublecortin (Dcx⁺) cells with the typical morphology for migrating neuroblasts appeared around the lesion (FIG. 1B). These neuroblasts, which were at least partly derived from the V-SVZ (FIG. 1C), were observed to be associated with Nestin⁺ fibers (FIGS. 1B to 1D). To label radial glial fibers specifically, Cre-encoding adenovirus (Ad-Cre) was injected into the cortical surface of P0 R26-tdTomato; Dcx-EGFP mice (Merkle et al., 2007, Science 317, 381-384) (FIGS. 1A and 1E). The tdTomato fluorescence clearly labeled fiber-bearing cells that expressed the radial glial cell markers Pax6, Nestin, and ErbB4 (Schmid et al., 2003, Proc. Natl. Acad. Sci. USA 100, 4251-4256). The cell bodies of these cells were observed in the V-SVZ and corpus callosum (CC), in addition to astrocytes and oligodendrocytes (FIG. 1E′). It was found that 55.5%±3.1% of the Dcx-EGFP⁺ neuroblasts migrated radially (toward the lesion) and that 96.0%±0.3% of these migrating neuroblasts were associated with tdTomato⁺Nestin⁺ radial glial fibers. Notably, 34.8±4.7% of the Dcx-EGFP⁺ neuroblasts aligned their whole cell body with radial glial fibers (FIGS. 1F and 1G). These results suggested that V-SVZ-derived neuroblasts that migrate radially toward the lesion after neonatal brain injury are associated with radial glial fibers.

N-Cadherin, a protein involved in regulating cell-cell adhesion, is involved in radial glia-guided neuroblast migration in the embryonic cortex (Kawauchi et al., 2010, Neuron 67, 588-602). The present inventors observed N-cadherin expression in both neonatal radial glial fibers and migrating neuroblasts after injury (FIG. 1D). To inactivate the function of N-cadherin in the radial glia, radial glial fibers were infected with an adenovirus vector encoding an inactive body (dominant negative form) of N-cadherin (DN-N-cadherin) and Cre at P0 (FIGS. 1A and 1F). The DN-N-cadherin expression in radial glia did not affect the morphology or density of the radial glia at 7 dpi. However, the proportion of neuroblasts associated with the DN-N-cadherin-expressing radial glial fibers was significantly lower than that in the control group (FIGS. 1F and 1G). Furthermore, the neuroblast density was significantly decreased in the DN-N-cadherin-virus-infected area and increased in the non-infected area, compared with those areas in control mice (FIG. 1H). This suggests that the neuroblasts preferred the radial glial fibers without DN-N-cadherin for their migration. Specific down-regulation of the N-cadherin expression in radial glia using an adenoviral knockdown ( KD) vector also decreased the proportion of neuroblasts associated with radial glial fibers and the neuroblast density (FIGS. 1I and 1J). These results suggested that radial glial N-cadherin is involved in the radial glial fiber-guided migration of neuroblasts after injury. The knockdown of FAK and L l-CAM, which are involved in radial glial fiber-guided neuroblast migration in the fetal period (Tonosaki et al., 2014, PLoS ONE 9, e86186;Valiente et al., 2011, J. Neurosci. 31, 11678-11691), did not affect the association of neuroblasts with radial glial fibers. Transmission electron microscopy (TEM) analyses revealed direct contacts between the neuroblasts and radial glial fibers, in which AJ-like electron-dense structures were occasionally observed (FIGS. 1K to 1K″, red arrows). The expression of DN-N-cadherin in the radial glia decreased the density of such structures and increased the proportion of irregular contacts, in which the cell membranes of neuroblasts and the cell membranes of radial glia were not parallel (FIGS. 1L to 1M, blue arrowheads). These observations suggested that the N-cadherin in radial glia is involved in forming the proper cell adhesion between radial glial fibers and migrating neuroblasts in the neonatal brain. Taken together, these results indicate that neonatal radial glia associate with the V-SVZ-derived neuroblasts that migrate toward the lesion after brain injury.

N-Cadherin Promotes RhoA Activation and Saltatory Movement of Neuroblasts Migrating Along Radial Glial Fibers

To examine whether the neuroblasts utilize radial glial fibers as a migratory scaffold toward a lesion, they were monitored by live imaging in cultured brain slices of R26-tdTomato; Dcx-EGFP mice at 4 to 5 dpi. Dcx-EGFP⁺ neuroblasts extended their leading process along tdTomato⁺ radial glial fibers and translocated their soma in the direction of the lesion in a saltatory manner (FIGS. 2A and 2A′). Neuroblasts migrating along DN-N-cadherin-expressing radial glia showed a significantly lower migration speed (FIGS. 2A and 2B) and frequent detachment from the radial glial fibers (FIGS. 2A, 2A′, and 2C). Consistent with the histological analyses (FIGS. 1G and 1I, the proportion of neuroblasts that was not attached to fibers was significantly increased in the DN-N-cadherin group (FIG. 2D). These results suggested that radial glial N-cadherin is involved in the efficient and continuous migration of neuroblasts along radial glial fibers toward a lesion.

The migration speed of neuroblasts is determined by the somatic stride length, somatic stride frequency, and length of the pause (resting phase) (Ota et al., 2014, Nat. Commun. 5, 4532). DN-N-cadherin expression in radial glia significantly decreased the somatic stride length and increased the duration of the resting phase and of each migration cycle in the neuroblast migration (FIGS. 2E to 2G). Collectively, these results suggested that the radial glial fiber-guided neuronal migration in the injured neonatal brain increases, through N-cadherin, the somatic stride length of neurons and the frequency of neurons' saltatory movement.

Since RhoA signaling in the proximally located swelling in the soma of migrating neuroblasts is known to promote their saltatory movement (Ota et al., 2014, Nat. Commun. 5, 4532), the present inventors next monitored their RhoA activity using fluorescent resonance energy transfer (FRET) imaging in an N-cadherin-Fc-coated stripe assay. The RhoA activity in the swelling of migrating neuroblasts was significantly increased when they migrated on an N-cadherin-containing scaffold (FIGS. 2H to 2J).

N-Cadherin can interact with various signaling molecules. Next, to examine whether the N-cadherin in neuroblasts was also involved in their migration on an N-cadherin-containing scaffold, N-cadherin knockdown plasmids were introduced into cultured neuroblasts, and their migratory behaviors were analyzed in a stripe assay (FIGS. 2K to 2P). The neuroblasts significantly increased their migration speed when they entered an N-cadherin-Fc stripe (FIGS. 2K and 2L). The increased migration speed appeared to be due to both an increased somatic stride length and a decreased time spent in the resting phase in each migration cycle (FIGS. 2K to 2O). This was consistent with the effects of DN-N-cadherin expression in radial glia (FIGS. 2A to 2G). When neuroblasts reached the border of an N-cadherin-Fc stripe, most of the control cells changed the direction of their leading process to remain on the N-cadherin-Fc-coated area. The percentage of cells that showed this behavior was significantly decreased by N-cadherin knockdown (FIGS. 2K and 2P). This suggests that the neuroblast's N-cadherin helps to maintain the directional neuroblast migration on an N-cadherin-containing scaffold. Taken together, these results suggested that N-cadherin promotes RhoA activation and saltatory movement of neuroblasts migrating along radial glial fibers.

N-Cadherin-containing Scaffold Promotes Recovery of Neurological Functions by Increasing V-SVZ-derived Neuronal Migration and Regeneration after Neonatal Brain Injury

The neonatal V-SVZ supplies newly produced mature neurons to the cerebral cortex under physiological conditions (Le Magueresse et al., 2011, J. Neurosci. 31, 16731-16747) and to the injured striatum and cortex after brain injury (Yang et al., 2007, Ann. Neurol. 61, 199-208; Yang et al., 2008, J. Comp. Neurol. 511, 19-33). Cryogenic injury of cerebral cortex increased the number of neuronal progenitor cells. The number of Dlx2⁺Dcx⁺ but not of Tbr2⁺Dcx⁺ neuroblasts was increased after injury. This suggests that GABAergic neuroblasts are recruited to the injured cortex. Furthermore, neonatal cryogenic injury of cerebral cortex significantly increased the number of EmGFP¹NeuN¹ mature neurons, which were mostly GAD67⁺ and less frequently Parvalbumin (PV)⁺ or Calretinin (CR)⁺. This indicates that they were V-SVZ-derived cortical interneurons. Over 60% of these neurons were located in cortical layers IV to VI. The number of V-SVZ-derived mature neurons in the cortex was significantly decreased by expressing DN-N-cadherin in the neonatal radial glia (FIGS. 3A to 3C). This suggests that radial glial fibers contribute to the migration and maturation of V-SVZ-derived neuroblasts in the injured neonatal cortex.

Next, to test whether an N-cadherin-containing artificial scaffold would promote V-SVZ-derived neuronal migration after brain injury, polyethylene terephthalate (PET) fibers and gelatin sponges conjugated with Fc or N-cadherin-Fc (control or N-cadherin fibers/sponges, respectively) were developed. The migration speed of the V-SVZ-derived neuroblasts increased when they made contact with the N-cadherin fibers and sponges in vitro (FIGS. 3D, 3E, and 3F). The present inventors then transplanted N-cadherin fibers or sponges into the injured cortex (FIG. 3F). While there was no significant difference in the density of Dcx⁺ neuroblasts between the control and N-cadherin fibers, the density of neuroblasts within the sponges was increased in the mice treated with the N-cadherin sponge (FIGS. 3G to 3I). This suggests that N-cadherin sponges support neuroblast migration more efficiently than do N-cadherin fibers in vivo, under the above experimental conditions.

To investigate whether N-cadherin sponges promote neuroblast migration in older brains that lack radial glia, the present inventors performed the cryogenic injury of cerebral cortex at P14 or 8 w and transplanted N-cadherin sponges (FIG. 3F). The number of neuroblasts reaching the lesion in the control-sponge groups was significantly smaller in the P14 and 8 w models compared with P2, supporting the concept that radial glial fibers are important scaffolds for neuroblast migration toward the lesion (FIGS. 3H and 3I). Consequently, however, the effect of N-cadherin sponge on the promotion of neuroblast migration was more obvious in the older brains, even though the absolute number of neuroblasts in the N-cadherin sponge was highest in the P2 model and decreased with age (FIGS. 3H and 3I).

Furthermore, N-cadherin sponge was transplanted at 10 dpi into the P2 injury model and the number of neuroblasts in the sponge was compared with that in the 3 dpi transplantation group (FIG. 3F). At 4 dpt, the density of neuroblasts was higher in the brains with transplantation at P5 than in those with transplantation at P12 (FIG. 3I). This suggests that early sponge transplantation had the most beneficial effect on neuroblast recruitment after neonatal brain injury.

To examine the effect of N-cadherin-sponge transplantation on neuronal regeneration, V-SVZ cells were labeled by electroporation, and their fate was analyzed at 28 dpi (FIGS. 3F, 3J, and 3K). The number of V-SVZ-derived NeuN⁺ mature neurons in and around the lesion was significantly greater in the mice treated with the N-cadherin sponge than in those treated with the control sponge (FIGS. 3J and 3K). Moreover, the proportion of V-SVZ-derived NeuN⁺ neurons in the upper cortical layers was significantly increased by transplanting N-cadherin sponge (FIG. 3K). These results suggested that the N-cadherin-containing scaffold promoted V-SVZ-derived neuronal regeneration after neonatal brain injury.

Finally, the effects of N-cadherin-sponge transplantation on functional recovery were investigated at 28 dpi. To analyze the spontaneous gait behaviors, we used CatWalk analyses. The brain injury caused a decrease in the contact area of the front paws (“Max contact area” and “Print area (total footprint area)”) and an increase in the width between the front paws (“Base of support”) (FIGS. 4A to 4C). Control-sponge transplantation did not worsen these gait behaviors (FIGS. 4A to 4C). This suggests that the sponge transplantation did not have any adverse effects. Notably, N-cadherin-sponge transplantation improved the defects in these gait parameters (FIGS. 4A to 4C). This suggests that the N-cadherin sponge promoted functional recovery in addition to neuronal regeneration after neonatal brain injury.

Next, we performed the foot-fault test (Barth et al., 1990, Behay. Brain Res. 39, 73-95). Cryogenic injury of cerebral cortex induced left-right asymmetry of the foot-fault ratio at 28 dpi in the P2 injury model. This was recovered by the transplantation of N-cadherin but not control sponge (FIG. 4D). N-Cadherin-sponge transplantation also led to a clear improvement in the neurological score in the P14 but not in the 8 w model (FIG. 4D). Thus, although neuroblast migration can be enhanced by N-cadherin sponge even in the adult brain, the time window for functional recovery appears to be more limited.

To further determine the contribution of V-SVZ-derived endogenous neuronal regeneration on functional recovery, Ad-Cre was intraventricularly injected into P0 neuron-specific enolase (NSE)-diphtheria toxin fragment A (DTA) mice (Imayoshi et al., 2008, Nat. Neurosci. 11, 1153-1161; Kobayakawa et al., 2007, Nature 450, 503-508), which eliminates neuronal progenies (FIGS. 4E to 4G). The improvement in the foot-fault ratio by N-cadherin-sponge transplantation was not observed in the Ad-Cre-infected NSE-DTA mice (FIG. 4H). Taken together, these results suggested that an N-cadherin-containing scaffold promoted the functional recovery after neonatal brain injury, and that V-SVZ-derived neuroblast regeneration contributed to this recovery.

What is claimed is:

1. A material for treatment of brain injury, comprising a carrier on which at least one selected from the group consisting of N-cadherin, a fusion protein containing an entire or partial region of N-cadherin, and a fusion protein containing an entire or partial region of a protein having homology to N-cadherin is immobilized or coated.

2. The material for treatment of brain injury according to claim 1, wherein the fusion protein containing an entire or partial region of N-cadherin, or the fusion protein containing an entire or partial region of a protein having homology to N-cadherin, has a homophilic binding capacity to N-cadherin.

3. The material for treatment of brain injury according to claim 1, wherein the fusion protein containing an entire or partial region of N-cadherin, or the fusion protein containing an entire or partial region of a protein having homology to N-cadherin, is a fusion protein containing a protein selected from the following (1) to (3):
(1) N-cadherin, or a protein having an amino acid sequence having an identity of not less than 90% to N-cadherin;
(2) an extracellular domain of N-cadherin, or a protein having an amino acid sequence having an identity of not less than 90% to an extracellular domain of N-cadherin; and
(3) a protein containing one or more of EC1 domain, EC2 domain, EC3 domain, EC4 domain, and EC5 domain of N-cadherin.

4. The material for treatment of brain injury according to claim 1, wherein the fusion protein is a fusion protein with an Fc region of an immunoglobulin.

5. The material for treatment of brain injury according to claim 1, wherein the carrier is a porous body.

6. The material for treatment of brain injury according to claim 1, wherein the carrier is a biomaterial or biocompatible polymer carrier.

7. The material for treatment of brain injury according to claim 1, wherein the carrier is a biomaterial porous body.

8. The material for treatment of brain injury according to claim 6, wherein the biomaterial is a protein or a polysaccharide.

9. The material for treatment of brain injury according to claim 6, wherein the biomaterial is gelatin or collagen.

10. The material for treatment of brain injury according to claim 1, wherein the carrier is a gelatin sponge.

11. A material for regeneration of brain neurons, comprising a carrier on which at least one selected from the group consisting of N-cadherin, a fusion protein containing an entire or partial region of N-cadherin, and a fusion protein containing an entire or partial region of a protein having homology to N-cadherin is immobilized or coated.

12. A method for treatment of brain injury, comprising transplanting the material for treatment of brain injury according to claim 1 into a brain.

13. The method for treatment of brain injury according to claim 12, wherein neuronal cells derived from pluripotent stem cells are transplanted into the brain together with the material for treatment, or after the transplantation of the material for treatment into the brain.

14. A method for regeneration of brain neurons, comprising transplanting the material for regeneration of brain neurons according to claim 11 into a brain.

15. The method for regeneration of brain neurons according to claim 14, wherein neuronal cells derived from pluripotent stem cells are transplanted into the brain together with the material for regeneration, or after the transplantation of the material for regeneration into the brain.


SEQUENCE LISTING (2 sequences)

SEQ ID NO: 1 - protein, 906 aa, Human
MCRIAGALRTLLPLLAALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKRDWVISPINLPENSRGPFPQELVRIRSDRDKNLSLRYSVTGPGADQPPTGIFIINPISGQLSVTKPLDREQIARFHLRAHAVDINGNQVENPIDIVINVIDMNDNRPEFLHQVWNGTVPEGSKPGTYVMTVTAIDADDPNALNGMLRYRIVSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVYRISGGDPTGRFAIQTDQNSNDGLVTVVKPIDFEANRMFVLTVAAENQVPLAKGIQHPPQSTATMSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYGIDPNAGPFAFDLPLSPVTIKRNWTITRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRMDERPIHAEPQYPVRSAAPHPGDIGDFINEGLKAADNDPTAPPYDSLLVFDYEGSGSTAGSLSSLNSSSSGGEQDYDYLNDWGPRFKKLADMYGGGDD

SEQ ID NO: 2 - protein, 906 aa, Mouse
MCRIAGAPRTLLPLLAALLQASVEASGEIALCKTGFPEDVYSAVLPKDVHEGQPLLNVKFSNCNRKRKVQYESSEPADFKVDEDGTVYAVRSFPLTAEQAKFLIYAQDKETQEKWQVAVNLSREPTLTEEPMKEPHEIEEIVFPRQLAKHSGALQRQKRDWVIPPINLPENSRGPFPQELVRIRSDRDKNLSLRYSVTGPGADQPPTGIFIINPISGQLSVTKPLDRELIARFHLRAHAVDINGNQVENPIDIVINVIDMNDNRPEFLHQVWNGSVPEGSKPGTYVMTVTAIDADDPNALNGMLRYRILSQAPSTPSPNMFTINNETGDIITVAAGLDREKVQQYTLIIQATDMEGNPTYGLSNTATAVITVTDVNDNPPEFTAMTFYGEVPENRVDVIVANLTVTDKDQPHTPAWNAAYRISGGDPTGRFAILTDPNSNDGLVTVVKPIDFETNRMFVLTVAAENQVPLAKGIQHPPQSTATVSVTVIDVNENPYFAPNPKIIRQEEGLHAGTMLTTLTAQDPDRYMQQNIRYTKLSDPANWLKIDPVNGQITTIAVLDRESPNVKNNIYNATFLASDNGIPPMSGTGTLQIYLLDINDNAPQVLPQEAETCETPEPNSINITALDYDIDPNAGPFAFDLPLSPVTIKRNWTINRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRLDERPIHAEPQYPVRSAAPHPGDIGDFINEGLKAADNDPTAPPYDSLLVFDYEGSGSTAGSLSSLNSSSSGGDQDYDYLNDWGPRFKKLADMYGGGDD